(19) United States
(12) Patent Application Publication
(10) Pub. No.: US 2013/0267606 A1
(43) Pub. Date: Oct. 10, 2013

(54) USE OF VITAMIN K FOR WEIGHT MAINTENANCE AND WEIGHT CONTROL

(71) Applicant: Cees Vermeer, Maastricht (NL)

(72) Inventor: Cees Vermeer, Maastricht (NL)

(73) Assignee: VITAK B.V., Maastricht (NL)

(21) Appl. No.: 13/994,518

(22) PCT Filed: Dec. 19, 2011
(86) PCT No.: PCT/EP11/73307

§ 371 Date: Jun. 14, 2013

(51) Int. Cl. A61K 31/122

(52) U.S. Cl. CPC A61K 31/122
USPC 514/681; 568/327

(57) ABSTRACT

Vitamin K is effective in counteracting (1) increase of body weight and body mass index (BMI), (2) accumulation of body fat mass and (3) accelerates weight loss during calory restriction or other life style interventions aiming weight reduction. A pharmaceutical composition or nutritional formulation comprising vitamin K is provided which can be used to combat overweight or obesity, either as a single, dedicated product or in combination with other slimming products or life style change

### FIELD OF THE INVENTION

The present invention is in the field of nutrition. In particular, the invention relates to increased vitamin K intake as a dietary intervention to improve weight maintenance, to decrease body fat mass, and maintain the body mass index (BMI) in the normal healthy range.

### BACKGROUND OF THE INVENTION

Vitamin K may occur in two different forms: K1 and K2. Whereas K1 comprises one single chemical structure (phylloquinone), K2 is a group name for the family of menaquinones (abbreviated as MK), which have in common a methylated naphthoquinone ring structure as the functional group, but which vary in the length of their polyisoprenoid side chain. The number of isoprenyl residues in the side chain may vary from 1 (in MK-1) to 13 in MK-13. In the generally adopted nomenclature n stands for the number of isoprenyl residues in MK-n. The different forms of vitamin K share the function as coenzyme for the posttranslational enzyme gammaglutamate carboxylase (GCCX), but substantial differences have been reported with respect to absorption, transport, and pharmacokinetics {Schurgers L J, Vermeer C. Biochim Biophys Acta 1570 (2002) 27-32). Whereas K1 is preferentially utilized by the liver, K2 vitamins (mainly the long chain menaquinones MK-7 through MK-10) are readily transported to extrahepatic tissues such as bone, arteries and adipose tissue.

The product of vitamin K action is the unusual aminoacid gammacarboxy-glutamic acid, abbreviated as Gla. Presently, 17 Gla-proteins have been discovered and in those cases in which their functions are known they play key roles in regulating important physiological processes including haemostasis, calcium metabolism and cell growth and survival {Berkner K L, Runge K W. J Thromb Haemostas 2 (2004) 2118-2132}. Since new Gla-proteins are discovered almost yearly {Viegas C S et al. Am J Pathol 175 (2009) 2288-2298}, it is to be expected that more Gla-protein-controlled processes will be identified in the near future. In all Gla-proteins the function of which is known, the Gla-residues are essential for the activity and functionality of these proteins whereas proteins lacking these residues are defective {Berkner K L, Runge K W. J Thromb Haemostas 2 (2004) 2118-2132}. The exquisite specificity with which Gla-domain structures facilitate interaction of vitamin K-dependent coagulation proteins with cell membranes is now becoming understood {Huang M et al. Nature Struct Biol 10 (2003) 751-756}. Likewise, it is well accepted that the Gla-residues of osteocalcin confer binding of the protein to the hydroxyapatite matrix of bone in a manner strongly suggestive of selectivity and functionality {Hoang Q Q. Nature 425 (2003) 977-980}.

The Gla-proteins involved in haemostasis are all synthesized in the liver: four blood coagulation factors (II, VII, IX, and X) and three coagulation inhibiting proteins (C, S, and Z). In the normal healthy population, vitamin K intake is sufficient to cover the requirements of the liver, so in healthy adults all coagulation factors are fully carboxylated. As will be detailed below, most extra-hepatic Gla-proteins are substantially under-carboxylated with 20-30% of the total antigen being present in the Gla-deficient (and hence inactive) state. Examples are the bone Gla-protein osteocalcin (OC) and the vascular Matrix Gla-Protein (MGP) {Knapen M H J et al. Ann Int Med 111 (1989) 1001-1005}{ Cranenburg E C M et al. Thromb Haemostas 104 (2010) 811-822}. Whereas the function of MGP as an inhibitor of soft tissue calcification is well understood {Schurgers L J et al. Thromb Haem 100 (2008) 593-603}, the function of OC has remained a matter of debate even 30 years after its discovery.

Lee and colleagues proposed the revolutionary hypothesis that the skeleton may act as an endocrine organ to regulate energy metabolism {Lee N K et al. Cell 130 (2007) 456-469; Ferron M et al. Proc Natl Acad Sci USA 105 (2008) 5266-5270}. A key concept of this hypothesis is that this regulation of energy metabolism is mediated by the bone-specific OC, which was invoked as a new hormone that facilitates β-cell proliferation, insulin secretion, and peripheral sensitivity to insulin. Evidence was also presented that the increased sensitivity in adipocytes was due to the stimulatory effect of OC on the secretion of adiponectin. OC was already known to be a major bone protein that is synthesized by osteoblasts during bone formation and contains three Gla-residues which are formed during a posttranslational vitamin K-dependent step {Berkner K L, Runge K W. J Thromb Haemostas 2 (2004) 2118-2132}. In contrast to the Gla proteins of the haemostatic system the vitamin K contained in most human diets is insufficient to support the full gamma-carboxylation of OC in bone. As a consequence, possibly combined with the inefficient transport of vitamin K to extra-hepatic organs, both carboxylated (cOC) and uncarboxylated (ucOC) species are synthesized and enter the circulation. Total circulating OC is a widely used bone formation marker, whereas conformation-specific assays for ucOC and cOC enable evaluation of the gamma-carboxylation status of newly synthesized OC {Vermeer C et al. Eur J Nutr 43 (2004) 325-335 }. Many nutritional studies have established that the ratio between circulating ucOC and cOC (UCR) is a useful biochemical marker of osteoblastic vitamin K status which quickly responds to changes in vitamin K intake {Binkley N C et al. Am J Clin Nutr 76 (2002)1055-1060; Iwamoto J et al. Nutr Res 29 (2009) 221-228}.

The most surprising aspect of the report of Lee at al. {Lee N K et al. Cell 130 (2007) 456-469} is not the evidence that OC may have more than one function (this is known for other vitamin K-dependent proteins) but their conclusion that the putative hormonal functions are mediated by the uncarboxylated form of OC rather than the carboxylated protein that was presumed to be inactive in glucose and fat metabolism. By the same group, a patent application (inventors: Ducy and Karsenty) was submitted in which undercarboxylated or uncarboxylated OC species (i.e. products resulting from vitamin K inadequacy) were claimed to promote energy metabolism (PCT/US07/20029, application Ser. No: 12/441,045). A logical consequence of this dependence on the gamma-carboxylation status of OC is that a low vitamin K intake will promote fat metabolism and weight loss, whereas a high vitamin K intake contributes to body mass increase and accumulation of adipose tissue.

Before the proposal of the hormone hypothesis for ucOC, a limited number of studies had suggested that an increased vitamin K intake has a potentially beneficial role in glucose homeostasis in rats and in young men {Sakamoto N et al. Int J Vitam Nutr Res 65 (1995)105-110; Sakamoto N et al. Diabetes Nutr Metab 12 (1999) 37-41; Sakamoto N et al. Clin. Nutr. 19 (2000) 259-263}. More recently, a study in the Framingham Offspring cohort by Yoshida et al {Yoshida M et al. Am. J. Clin. Nutr. 88 (2008) 210-215} showed that higher phylloquinone intakes were associated with greater insulin sensitivity and more favourable glycemic status among non-diabetic men and women. The same authors found that after 3 years of vitamin K1 supplementation, the progression of insulin resistance was reduced in older men, but not in women. No correlation was found between plasma vitamin K1 concentrations and percent body fat in either men or women {Yoshida M et al. Diabetes Care 31 (2008) 2092-2096}. Others found that 1 year of vitamin K1 supplementation did not alter glucose metabolism in a group of healthy postmenopausal women {Kumar R et al. Am J Clin Nutr 92 (2010) 1528-1532}. Recently, Shea et al demonstrated that adipose tissue contains high concentrations of vitamin K and that increased adiposity was associated with poor vitamin K status in the elderly, probably because the adipose tissue absorbs vitamin K (which is a fat-soluble vitamin) from the circulation {Shea M K et al. J Nutr 140 (2010) 1029-34}.

WO 02/01969 (Horrobin D F) discloses that vitamin K in combination with at least one essential fatty acid (EFA) as the essential ingredients may favourably influence a number of conditions including diabetes and obesity. The examples clearly show that in vitamin K insufficiency the efficacy of EFA is much less than in vitamin K sufficiency. Horrobin is silent, however, on the effect of vitamin K alone which must have a completely different mechanism since it is independent of EFA.

EP 1889613 A1 (Inoue S et al.) discloses that vitamin K has a beneficial effect in combination with a group of drugs, known as PPARδ inhibitors, which are suggested for the treatment of muscular disorders. Additionally, the use of PPARδ inhibitors for treating a variety of conditions including obesity is proposed. It is proposed that these conditions aggravate because PPARδ inhibitors induce a state of vitamin K deficiency leading to impaired blood coagulation. So treatment with a combination of vitamin K and PPARδ inhibitors has a better effect than treatment with PPARδ inhibitors in the absence of vitamin K. The investigators are silent, however, on the role of vitamin K in the absence of PPARδ inhibitors.

Moriya, Naktsuji and Sakoda (JP 2006 290772 A; XP-002625433) teach that extremely high doses of vitamin K2 (62.5 mg/kg body weight per day) may be protective in an animal model for auto-immune disease. Rats were made ill by injection with myelin basic protein and Freund's tuberculus bacillus toxin and the investigators noticed that animals in the absence of vitamin K2 were more ill and lost more weight than the vitamin K2-treated animals. The dose of vitamin K2 (62.5 mg/kg body weight) compares with 500 milligrams per day in humans and is well above doses that have ever been used in humans (generally 15-100 micrograms per day, in exceptional cases up to 45 milligrams per day).

Pan Y and Jackson R T, J Am Coll Nutr. 28 (2009) 369-379 describe a population-based study in which an association between vitamin K1 (phylloquinone) intake and low body weight was found. As stated by the authors, the inverse association between K1 and body weight is probably caused by the vitamin K1-rich diet, which was shown in other studies to be of lower caloric value and more heart-healthy and rich in vegetables {Braam et al, J Am Diet Assoc. 104 (2004) 1410-1414}. The alternative explanation, i.e. via activation of fat metabolism by the vitamin K-dependent protein osteocalcin cannot be true, since this theory (presented in references 10 and 11 of their paper) predicts that uncarboxylated osteocalcin (vitamin K-insufficiency) promotes fat metabolism, whereas carboxylated osteocalcin (vitamin K sufficiency) is associated with high body weight. Obviously, a direct effect of vitamin K on body weight can only be concluded from intervention studies, but an alternative would be to investigate the potential association between vitamin K2 (which is known to be present in unhealthy high fat diets) and body weight. Only if the investigators would have found an inverse association between both vitamin K1 and K2 intake and body weight, this would be suggestive for a direct effect of vitamin K. Since vitamin K2 was not included in the study, it must be assumed that the observed effect was caused by the heart-healthy low-calorie diet.

Presently, no data have been disclosed about an association of vitamin K status with body weight and neither data have been disclosed about effects of supplemental vitamin K intake on weight gain or loss, or on the increase or decrease of body fat mass.

### SUMMARY OF THE INVENTION

In one aspect of this invention, a pharmaceutical or nutritional formulation is provided comprising vitamin K to increase the vitamin K status in a subject, for use in a method for the treatment or prevention of overweight in subjects with a tendency to increase body weight, with the proviso that said pharmaceutical or nutritional formulation does not contain an essential fatty acid (EFA).

In another aspect of the present invention, increased vitamin K intake is provided as a method for weight maintenance in subjects with a tendency to increase body weight, for instance in postmenopausal women, subjects with low physical activity, subjects with an unhealthy lifestyle, or children.

In still another aspect of this invention, increased vitamin K intake is provided as a method sustained weight reduction following other weight reduction measures such as increased physical activity and/or more healthy dietary habits, or diets/regimens aiming rapid weight loss.

In still another aspect of this invention, increased vitamin K intake is provided as a method for weight reduction in overweight or obese people.

In still another aspect of this invention, increased vitamin K intake is provided as a method to be combined with other weight reducing measures.

In still another aspect of this invention, increased vitamin K intake is provided as a method to help decrease body fat mass and improve hip waist ratio.

In still another aspect of this invention, increased vitamin K intake is also provided as a method to help combat overweight and obesity.

These and other aspects of the present invention will be more fully outlined in the detailed description which follows.

### BRIEF DESCRIPTION OF THE DRAWINGS

FIG. 1: Changes in weight and BMI during treatment with vitamin vitamin K₂ (MK-4, 45 mg/day) or placebo in 325 apparently healthy postmenopausal women (age range: 55-75 years) during a 3-year intervention study. Error bars indicate SEM. The increases within the placebo group were statistically significant after 3 years (within-group paired data analysis), as were the differences between the vitamin K and the placebo group (unpaired analysis).

FIG. 2: Changes in BMI during treatment with vitamin K₁ 1 mg/day) or placebo 120 apparently healthy but overweight subjects (age range: 35-60 years, 52 men, 68 women) during a 2-year intervention trial. Error bars indicate SEM. The difference between the vitamin K-treated and placebo-treated group was not significant after completing the weight reduction intervention period, but during the subsequent 18 months the weight gain in the vitamin K-treated group was significantly less than in the placebo group (p<0.05, unpaired analysis).

### DETAILED DESCRIPTION OF THE INVENTION

Vitamin K is a group name for compounds sharing a methylated naphthaquinone ring and may be subdivided into vitamin K1 (phylloquinone), vitamin K2 (menaquinone) and vitamin K3 (menadione). Vitamin K2 may be subdivided further into a series of menaquinones which differ from each other with respect to the number of isoprenyl residues in an aliphatic side chain attached to the naphthoquinone rings. These menaquinones are commonly referred to as MK-n, where n relates to the number of isoprenyl residues. So MK-1 contains 1 isoprenyl group and MK-10 contains 10 isoprenyl residues. The most abundant menaquinones in the human diet are MK-4, MK-7, MK-8, MK-9 and MK-10. In nature, also MK-11 through MK-14 have been identified.

As used herein, the term “vitamin K” comprises each and every form of vitamin K1, vitamin K2 and vitamin K3, and combinations thereof, in the broadest sense, including individual forms of menaquinones and mixtures of menaquinones, unless stated otherwise. Particularly preferred for the purpose of the present invention are MK-4, MK-7, MK-8, MK-9 and MK-10, and mixtures thereof.

As used herein, overweight people are defined as those with a body mass index (BMI) between 25 and 30 kg/m². Obesity is defined herein as a body mass index>30 kg/m².

The present invention is based on the surprising discovery that different forms of vitamin K have weight reducing and fat-lowering properties if given on top of a normal healthy diet. Vitamin K is a cofactor for GCCX, an enzyme that carboxylates specific glutamate residues into gammacarboxyglutamate (Gla) in a posttranslational step. Presently, the class of Gla-proteins contains 17 members, but almost yearly new Gla-proteins are discovered, and more physiological functions are discovered to be “vitamin K-dependent”. A typically Western type of diet contains insufficient vitamin K to fully carboxylate all Glu residues into Gla residues in the Gla-proteins formed outside the liver. As a consequence, non-carboxylated species of these proteins are formed in the normal, “healthy” human population. These non-carboxylated, or under-carboxylated, proteins have no biological activity.

More specifically, the present invention is based on the surprising discovery that high vitamin K status or high dietary vitamin K intake results in a decreased tendency to accumulate body fat mass and in a decreased tendency to increase body weight. The underlying mechanism could not be attributed directly to adiponectin, osteocalcin or other known Gla-proteins, so we conclude that the favorable effect of vitamin K on body weight is brought about via unknown mechanisms or even presently unknown Gla-proteins. We have demonstrated that circulating cOC (as a marker for high vitamin K status) was inversely and independently associated with body mass index (BMI), waist-circumference (W-circ), waist-hip ratio (WHR), fat mass (FM)and fat mass of the trunk FMT). Moreover, high values of UCR (suggestive of a poor vitamin K status) were associated with high BMI and high fat mass. Supplementation with vitamin K (either K1 or K2) did not affect circulating adiponectin concentrations and did not lead to an increase in BMI. On the contrary, we observed an opposite trend such that the BMI remained unchanged in vitamin K-supplemented groups, but significantly increased in the placebo groups. To our knowledge, no vitamin K intervention studies have been published in which body weight or BMI have been included as a clinical endpoint.

The strong inverse association we found between adiponectin and fat mass, especially with abdominal fat mass (W-circ, FMT and FMT %) has been reported by many others {Ziemke F et al. Am J Clin Nutr 91 (2010) 258S-61S}. Here we demonstrate that cOC and not ucOC was inversely associated with the fat mass indices suggesting that the vitamin K status was lower (e.g. high UCR) in subjects with higher weight, W-circ and fat mass. Subjects with a high degree of OC carboxylation were leaner and had less body fat than those with lower OC carboxylation.

Gla-residues are important for the binding of Gla-proteins to extracellular surfaces (phospholipids or hydroxyapatite), and since such binding does not seem to be associated with the postulated hormone function of osteocalcin, it seems at least plausible that domains outside the Gla-domain are important for its regulatory function in fat metabolism. This would imply that both carboxylated and uncarboxylated osteocalcin species possess a hormone function. It should be kept in mind, however, that in the presence of calcium ions carboxylated osteocalcin adopts a tertiary structure that is completely different from that of uncarboxylated osteocalcin, which may have implications for the molecular structure outside the Gla-domain and for the relative hormonal activity of ucOC and cOC. Our data suggest that cOC is the most active form in this respect. Another possible explanation for the effect of vitamin K on the maintenance of weight is that it acts directly on cellular functions, which are independent of gamma-carboxylation. Takeuchi and coworkers {Takeuchi Y et al. Bone 27 (2000) 769-776} presented evidence that MK-4 but not phylloquinone inhibited adipogenesis but stimulated osteoblastic differentiation in vitro. This is in line with a body of evidence that MK-4 has direct effects on a variety of cellular processes and pathways {Shearer M J et al. Thromb Haemostas 100 (2008) 530-547} but does not explain the effects of other forms of vitamin K.

In certain embodiments, the present invention includes the use of vitamin K-containing supplements or vitamin K-rich foods or vitamin K-enriched foods on top of the normal diet or partly replacing the normal diet; this is particularly helpful in subjects with a normal weight (BMI 20-25) or who are slightly overweight (BMI 25-30), and who are at risk of increasing body weight. Examples are women shortly after the menopause (average body weight increase about 0.5 kg/year during the first 10 years after the menopause), subjects who stop or decrease their physical activity, for instance because of change of life style or trauma, subjects with a too high carbohydrate or fat intake, and children who have an increasing tendency to become overweight at young age.

In another embodiment the present invention includes the use of vitamin K supplements or vitamin K-rich or vitamin K-enriched foods as a method to sustain the weight reduction that has been attained by other weight reduction measures such as increased physical activity and/or more healthy dietary habits, slimming products, or diets/regimens aiming rapid weight loss. At high vitamin K intake on a long-term basis, the target body weight will be maintained for longer periods than at low vitamin K intake.

In still another embodiment the present invention includes the use of vitamin K supplements or vitamin K-rich or vitamin K-enriched foods as a method to induce body weight reduction and fat mass reduction in overweight or obese people. This method is especially suited for the slow, long-term strategy with a weight loss of approximately 1 kg per year. If sustained for 20-30 years, a significant weight reduction may be attained.

In still another embodiment the present invention includes the use of vitamin K supplements or vitamin K-rich or vitamin K-enriched foods in combination with other weight reduction measures such as increased physical activity and/or more healthy dietary habits, slimming products, or diets/regimens aiming rapid weight loss. The combination of these measures with increased vitamin K intake will accelerate weight loss and thus allow reaching the target weight within shorter time.

In still another embodiment the present invention includes the use of vitamin K supplements or vitamin K-rich or vitamin K-enriched foods is provided as a method to help decrease body fat mass and improve hip waist ratio.

In still another embodiment the present invention includes the use of increased vitamin K intake is also provided as a method to help combat overweight and obesity.

In still another embodiment the present invention includes the use of vitamin K supplements or vitamin K-rich or vitamin K-enriched foods.

In still another embodiment the present invention includes the preparation of vitamin K supplements or vitamin K-rich or vitamin K-enriched foods to be used for weight maintenance or weight reduction in humans, allowing a preferred supplemental dose of vitamin K1 between 1 and 5000 micrograms per day, or a more preferred dose of vitamin K1 between 10 and 2000 micrograms per day, or a still more preferred dose of vitamin K1 between 50 and 1000 micrograms per day, or a most preferred dose between 100 and 500 micrograms per day.

In still another embodiment the present invention includes the preparation of vitamin K supplements or vitamin K-rich or vitamin K-enriched foods to be used for weight maintenance or weight reduction in humans, allowing a preferred supplemental dose of vitamin K2, preferably MK-7, MK-8, or MK-9, between 1 and 5000 micrograms per day or a more preferred dose of vitamin K2 between 10 and 1000 micrograms per day, or a still more preferred dose of vitamin K2 between 20 and 500 micrograms per day, or a most preferred dose between 50 and 500 micrograms per day.

The potential application of the present invention will be demonstrated in the following examples.

### EXAMPLE 1

Design: Data on body composition, osteocalcin and adiponectin measurements was obtained from a cohort of 380 apparently healthy postmenopausal women (age range: 55-65 years). Exclusion criteria were: BMI>30 kg/m², a medical history or use of drugs known to interfere with vitamin K-, calcium- and/or glucose-metabolism.

Methods: Body height was measured using a wall-mounted stadiometer. Body weight was measured to the nearest 0.1 kg with participants wearing light clothing and no shoes. Body mass index (BMI) was calculated as body weight (kg) divided by the square of height (m²). Waist-to-hip ratio (WHR) was calculated as the waist circumference (W-circ in cm) divided by the hip circumference (H-circ in cm), with a precision of 0.5 cm. Whole body fat mass (FM) and fat mass of the trunk (FMT), both expressed in kg, were measured by dual X-ray absorptiometry (DXA; Discovery A, Hologic, Bedford Mass.), using the whole-body absorptiometry software package. Blood was taken by venipuncture after an overnight fasting period. Blood was collected between 8 and 11 am. Serum was prepared by centrifugation and stored at −80° C. until analysis. Commercially available ELISA tests were used to determine serum ucOC and cOC (Takara Shuzo Co Ltd., Shiga, Japan). UCR was calculated as the ratio between ucOC and cOC and is used as a sensitive marker for bone vitamin K status with elevated values of UCR indicating a low vitamin K status. Total serum adiponectin was measured by an ELISA obtained from BioSource, Europe SA.

Results: As expected, serum adiponectin was inversely associated with body weight but not with body height. Adiponectin was also inversely associated with indices for fat distribution of the trunk (W-circ, WHR, FMT). Similar, but much stronger negative associations were found between the body composition variables and cOC but not for ucOC. As might be expected from the inverse correlation of body composition variables with cOC, the marker of bone vitamin K status UCR was positively associated with body composition indices. See Table 1 below.

TABLE 1
Pearson correlation coefficients between body composition
variables and adiponectin, ucOC, cOC and UCR

| | log(Adiponectin) | | ucOC | | cOC | | log(UCR) | |
|---|---|---|---|---|---|---|---|---|
| | r | P | r | P | r | P | r | P |
| Age | 0.171 | 0.012 | 0.060 | 0.381 | 0.025 | 0.712 | 0.043 | 0.525 |
| Body weight | −0.189 | 0.005 | 0.041 | 0.550 | −0.255 | <0.0001 | 0.174 | 0.010 |
| Body height | −0.091 | 0.183 | 0.044 | 0.517 | −0.007 | 0.916 | 0.051 | 0.451 |
| BMI | −0.165 | 0.015 | 0.020 | 0.766 | −0.296 | <0.0001 | 0.171 | 0.011 |
| W-circ | −0.317 | <0.0001 | 0.072 | 0.294 | −0.337 | <0.0001 | 0.247 | <0.0001 |
| H-circ | −0.039 | 0.567 | 0.038 | 0.573 | −0.260 | <0.0001 | 0.184 | 0.007 |
| WHR | −0.405 | <0.0001 | 0.068 | 0.316 | −0.216 | 0.001 | 0.171 | 0.012 |
| FM | −0.135 | 0.048 | 0.052 | 0.445 | −0.324 | <0.0001 | 0.213 | 0.002 |
| FMT | −0.292 | <0.0001 | 0.018 | 0.786 | −0.378 | <0.0001 | 0.220 | 0.001 |

Correlation coefficients are given with corresponding p-value. P-value <0.05 is considered to be statistically significant. Adiponectin and UCR were log-transformed to normalize the distribution.
Abbreviations used: BMI, body mass index; ucOC, uncarboxylated osteocalcin; cOC, carboxylated osteocalcin; UCR, ucOC/cOC ratio; W-circ, waist-circumference; H-circ, hip-circumference; WHR, waist-to-hip ratio; FM, total fat mass; FMT, fat mass of the trunk.

No associations were found between ucOC, cOC or UCR and adiponectin in an unadjusted model. After controlling for confounders (age, BMI and gender) the outcomes remained the same.

### Conclusions:

(1) in the non-supplemented population, vitamin K status had no effect on circulating adiponectin levels;
(2) high circulating cOC (indicative for high vitamin K status) was correlated with low body weight, low BMI and low fat mass.

### EXAMPLE 2

Design: Data of circulating osteocalcin and adiponectin was collected from a vitamin K₂ (MK-7) dose-response study in which 24 healthy men and 26 healthy premenopausal women (age range: 25-45 years) were randomized into 5 groups of 10 subjects each. These groups were treated with 0, 45, 90, 180 or 360 μg MK-7 per day for 12 weeks. Exclusion criteria were: BMI>30 kg/m², a medical history or use of drugs known to interfere with vitamin K-, calcium- and/or glucose-metabolism.

Methods: Blood was taken by venipuncture after an overnight fasting period. Blood was collected between 8 and 11 am. Serum was prepared by centrifugation and stored at −80° C. until analysis. Commercially available ELISA tests were used to determine serum ucOC and cOC (Takara Shuzo Co Ltd., Shiga, Japan). UCR was calculated as the ratio between ucOC and cOC and is used as a sensitive marker for bone vitamin K status with elevated values of UCR indicating a low vitamin K status. Total serum adiponectin was measured by an ELISA obtained from BioSource, Europe SA. Linear regression analysis was used to examine the relationship between the outcome variables ucOC, cOC and UCR and the independent variable adiponectin. These analyses were controlled for potential confounding variables such as age, BMI and gender. Moreover, the effect of increasing amounts of MK-7 on adiponectin, ucOC, cOC and UCR was determined in separate linear models. Serum concentrations of adiponectin, ucOC, cOC and UCR after 12 weeks of supplementation were the outcome variables and the MK-7 dose (0-360 μg) was the independent continuous variable, adjusted for the baseline values of adiponectin, ucOC, cOC, UCR, age, BMI and gender.

Results: No associations were found between ucOC, cOC or UCR and adiponectin in the unadjusted model. This outcome remained unchanged after controlling for confounders (age, BMI and gender), but a significant decrease was observed of circulating ucOC and of the ucOC/cOC ratio, demonstrating a biological effect of vitamin K even at a daily dose of 45 μg/day.

### Conclusions:

(1) at nutritional levels supplemental vitamin K2 (as MK-7) does not affect circulating adiponectin.
(2) an increase of vitamin K intake at doses of 45 μg/day resulted in a significant effect on biomarkers for vitamin K status, and this result was even obtained in small study cohorts.

### EXAMPLE 3

Design: 325 apparently healthy postmenopausal women (age range: 55-75 years) were recruited to a placebo controlled randomized trial to investigate the influence of vitamin K₂ (MK-4, 45 mg/day) on BMI and serum markers during a 3-year intervention study. Exclusion criteria were: BMI>30 kg/m², a medical history or use of drugs known to interfere with vitamin K-, calcium- and/or glucose-metabolism.

Methods: Body height was measured using a wall-mounted stadiometer. Body weight was measured to the nearest 0.1 kg with participants wearing light clothing and no shoes. Body mass index (BMI) was calculated as body weight (kg) divided by the square of height (m²). Waist-to-hip ratio (WHR) was calculated as the waist circumference (W-circ in cm) divided by the hip circumference (H-circ in cm), with a precision of 0.5 cm. Blood was taken by venipuncture after an overnight fasting period. Blood was collected between 8 and 11 am. Serum was prepared by centrifugation and stored at −80° C. until analysis. Commercially available ELISA tests were used to determine serum ucOC and cOC (Takara Shuzo Co Ltd., Shiga, Japan). UCR was calculated as the ratio between ucOC and cOC and is used as a sensitive marker for bone vitamin K status with elevated values of UCR indicating a low vitamin K status. Total serum adiponectin was measured by an ELISA obtained from BioSource, Europe SA. Within group differences were tested by the Paired-Sample T test or by the Wilcoxon test (UCR and adiponectin). All analyses were considered to be statistically significant at P-values<0.05. Values are presented as means±SD. Statistical analysis was performed using the statistical package (SPSS vs 17.0 Corp, Chicago, Ill.).

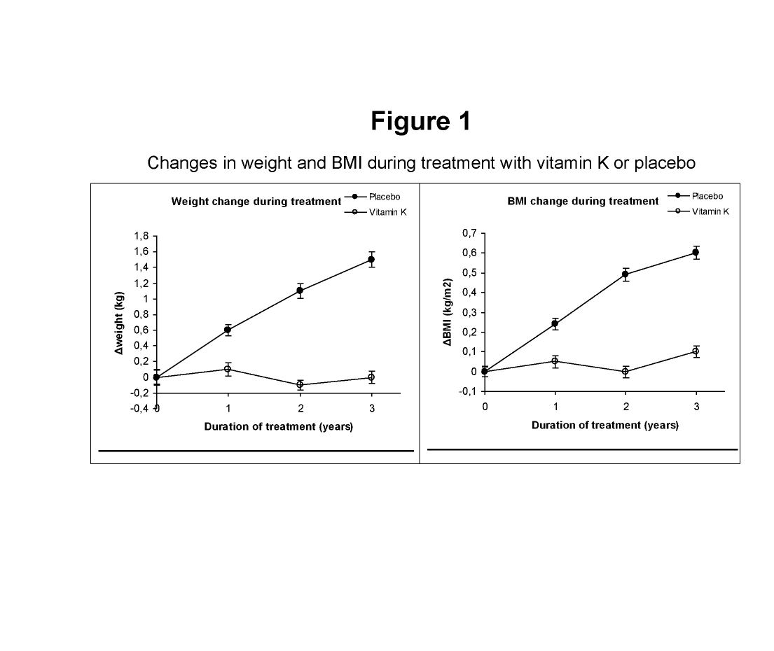

Results: As expected, ucOC significantly decreased after 3 years high-dose MK-4 treatment from 3.2±1.9 ng/ml to 0.8±1.0 ng/ml (P<0.0001), whereas ucOC in the placebo group remained unchanged after 3 years (3.0±1.6 ng/ml; P=0.769). cOC had significantly increased in the MK-4 group from 6.4±2.5 ng/ml to 6.9±2.3 ng/ml (P=0.046) and decreased in the placebo group from 6.8±2.2 ng/ml to 4.2±1.4 ng/ml (P<0.0001). Values of UCR in the placebo group increased with 77±81% from 0.48±0.30 at baseline to 0.76±0.52 (P<0.0001). I In the MK-4 group the values had decreased with 74±22% from 0.54±0.35 to 0.12±0.15 (P<0.0001). It is noteworthy that no significant difference was observed between the circulating adiponectin in both study arms, neither at baseline (placebo: 14.4±9.5 μg/ml, MK-4: 14.2±9.7 μg/ml; P=0.562) nor after 3 years of treatment (placebo: 13.2±6.6 μg/ml, MK-4: 13.1±8.3 μg/ml; P=0.224). Between-group analysis revealed that the changes after 3 years in ucOC, cOC and UCR differed significantly (P<0.0001), whereas changes in adiponectin did not differ (P=0.224). With respect to body weight and BMI it was found that after 3 years supplementation with placebo the average body weight had increased significantly from 71.8±1.0 kg to 73.3±1.2 kg and the BMI had increased from 27.3±0.3 kg/m² to 27.9±0.4 kg/m² (P=0.0001); in the MK-4 group, on the other hand, the average body weight had remained constant (70.3±0.9 kg), and the BMI had increased non-significantly from 27.1±0.3 kg/m² to 27.2±0.3 kg/m² (P=0.463). In the unpaired t-test the difference in response to treatment was statistically significant at P<0.001 after 3 years (see also FIG. 1).

### Conclusions:

(1) at high intakes, supplemental vitamin K2 (as MK-4) does not affect circulating adiponectin levels;
(2) in a group at risk for body weight increase, high vitamin K2 intake as the only intervention resulted in weight maintenance, whereas in the placebo group a significant increase of body weight was observed.

### EXAMPLE 4

250 apparently healthy postmenopausal women (age range: 55-75 years) were recruited in a placebo controlled 3-year randomized trial to investigate the influence of vitamin K₂ (MK-7, 180 μg/day) on body weight and BMI. Exclusion criteria were: BMI>30 kg/m², a medical history or use of drugs known to interfere with vitamin K-, calcium- and/or glucose-metabolism. Body height was measured using a wall-mounted stadiometer. Body weight was measured to the nearest 0.1 kg with participants wearing light clothing and no shoes. Body mass index (BMI) was calculated as body weight (kg) divided by the square of height (m²). Statistical analysis was performed using the statistical package (SPSS vs 17.0 Corp, Chicago, Ill.). After 3 years supplementation with placebo the average body weight had increased significantly from 66.7 to 69.3 kg and the BMI had increased from 25.4±0.81 kg/m² to 26.4±2.5 kg/m² (P=0.0001); in the MK-4 group, on the other hand, the average body weight had decreased non-significantly from 66.9 to 67.4 kg, and the BMI from 25.2±2.8 kg/m² to 25.4±3.0 kg/m² (P=0.463). In the unpaired t-test the difference in response to treatment was statistically significant at P<0.005 after 3 years.

### Conclusions:

(1) at nutritionally relevant intakes, supplemental vitamin K2 (as MK-7) resulted in maintenance of body weight and BMI, whereas an unfavourable increase in body weight and BMI was observed in the participants receiving placebo.

### EXAMPLE 5

120 apparently healthy but overweight subjects (age range: 35-60 years, 52 men, 68 women) were recruited in a placebo controlled 2-year randomized trial to investigate the influence of vitamin K1 (1 mg/day) on body weight and BMI during and after a 6-month intervention aiming significant weight reduction. At the start of the study participants were randomized to receive either placebo or vitamin K for 24 months. All participants received extensive dietary counselling and were motivated to increase their physical exercise during the first 6 months of the study, with personal contact once weekly. After this initial period, counselling was stopped, but vitamin K/placebo treatment was continued for another 18 months, while participants visited our institute every half year. Inclusion criteria were: apparently healthy, BMI between 25 and 35 kg/m², Exclusion criteria were: a medical history or use of drugs known to interfere with vitamin K-, calcium- and/or glucose-metabolism. Body height was measured using a wall-mounted stadiometer. Body weight was measured to the nearest 0.1 kg with participants wearing light clothing and no shoes. Body mass index (BMI) was calculated as body weight (kg) divided by the square of height (m²). Statistical analysis was performed using the statistical package (SPSS vs 17.0 Corp, Chicago, Ill.).

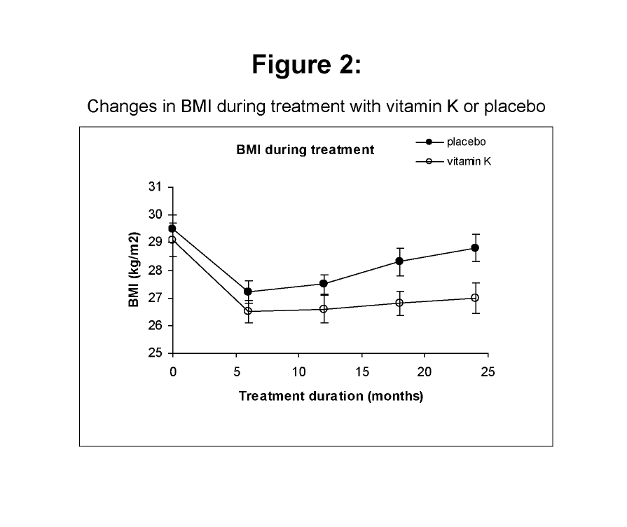

During the first 6 months the average body weight and BMI of the participants in both groups decreased (see FIG. 2), with a non-significant better performance in the vitamin K group. During the following 18 months, both body weight and BMI steadily increased in the placebo group because the new, healthy life style was discontinued partly or in whole. If analyzed on an individual basis only 11 subjects (5 men, 6 women) maintained or even lost further weight during the second phase of the study, whereas 33 participants had returned to their starting weight or higher. In the vitamin K-group the average weight gain was significantly lower (p<0.05) than in the placebo group, since 28 participants did not show significant weight gain during the second phase of the study, and only 7 had returned to their starting weight.

### Conclusions:

(1) adopting a healthier life style may lead to more pronounced weight reductions if combined with high vitamin K intake;
(2) weight reductions attained by dietary interventions are more easily sustained at high than at low vitamin K status.

These examples demonstrate that vitamin K can be used as an active ingredient to control body weight. Relatively high doses were used to demonstrate the proof of principle (180 μg/day-45000 μg/day). However, the effect on biomarkers for vitamin K status was also clear at much lower intake levels (up to 45 μg/day). Population-based studies have even shown that still lower doses may have dramatic clinical effects. Geleynse et al {J. Nutr. 134 (2004) 3100-3105} showed that subjects with a habitual dietary vitamin K2 intake of 35 μg/day and higher had 40% lower risk of cardiovascular mortality than those with a habitual intake of 12.5 μg/day. Hence the difference of 22.5 μg/day accounts for a strong mortality risk reduction. Gast et al {Nutrition, Metabolism & Cardiovascular Diseases 19 (2009) 504-510} studied a much larger cohort and by the increased power they were able to demonstrate that each 10 μg/day of incremental K2 intake is associated with a cardiovascular risk reduction of 9%. A statistically significant contribution could even be calculated per 1 μg/day of the long-chain menaquinones (K2 isomers). Although in small-scale intervention studies these low doses will not readily result in measurable effects, the population-based studies demonstrate that—if taken on a life-long basis—even very low doses of vitamin K may result in clinically significant beneficial outcomes.

1. A pharmaceutical or nutritional formulation comprising vitamin K to increase the vitamin K status in a subject, for use in a method for the treatment or prevention of overweight in subjects with a tendency to increase body weight, with the proviso that said pharmaceutical or nutritional formulation does not contain an essential fatty acid (EFA).

2. A pharmaceutical or nutritional formulation comprising vitamin K according to claim 1, wherein said treatment or prevention is to improve or facilitate weight reduction in overweight subjects.

3. A pharmaceutical or nutritional formulation comprising vitamin K according to claim 1, wherein said treatment or prevention is to reduce body fat mass or improve waist-hip ratio in subjects with a tendency to accumulate body fat.

4. A pharmaceutical or nutritional formulation comprising vitamin K according to claim 1, wherein the pharmaceutical or nutritional formulation is a formulation for oral use.

5. A pharmaceutical or nutritional formulation comprising vitamin K according to claim 1, wherein the vitamin K is contained in a food or beverage product or a dietary supplement.

6. A pharmaceutical or nutritional formulation comprising vitamin K according to claim 1, wherein the vitamin K comprises vitamin K₁ (phylloquinone), vitamin K₂ (menaquinone), vitamin K₃ (menadione) or a combination thereof.

7. A pharmaceutical or nutritional formulation comprising vitamin K according to claim 1, wherein the daily dosage of vitamin K to be administered to a subject is a daily dosage of vitamin K1 between 1 μg and 5000 μg, or between 10 μg and 2000 μg, or between 50 μg and 1000 μg, or between 100 μg and 500 μg.

8. A pharmaceutical or nutritional formulation comprising vitamin K according to claim 1, wherein the daily dosage of vitamin K to be administered to a subject is a daily dosage of vitamin K2, MK-7, MK-8, or MK-9, between 1 μg and 5000 μg, or between 10 μg and 1000 μg, or between 20 μg and 500 μg, or between 50 μg and 500 μg.

9. A pharmaceutical or nutritional formulation comprising vitamin K according to any claim 1, wherein said formulation is administered to a subject in combination with at least one of a slimming product, dietary intervention or other measures aiming at weight control or weight reduction.

10. A pharmaceutical or nutritional formulation comprising vitamin K according to claim 1, wherein the pharmaceutical or nutritional formulation is used to improve or facilitate weight maintenance after completing a slimming diet.

11. A pharmaceutical or nutritional formulation comprising vitamin K according to claim 1, wherein subjects with a tendency to increase body weight include postmenopausal women, subjects with low physical activity, subjects with an unhealthy lifestyle, and children.
